(19) United States
(12) Patent Application Publication
(10) Pub. No.: US 2021/0018437 A1
(43) Pub. Date: Jan. 21, 2021

(54) METHOD OF AND APPARATUS FOR PRODUCING AN IMAGE ON A DISPLAY SCREEN

(71) Applicant: Thomas Kerber, Stoney Creek (CA)

(72) Inventor: Thomas Kerber, Stoney Creek (CA)

(21) Appl. No.: 16/514,686

(22) Filed: Jul. 17, 2019

(51) Int. Cl. G01N 21/64; A61B 5/00; G01N 33/574

(52) U.S. Cl. CPC G01N 21/6428; G01N 2021/6439; G01N 33/57484; A61B 5/0071

(57) ABSTRACT

A method of producing an image on a display screen comprises the steps of generating at least one wavelength of light in a selected wavelength band of red light in the wavelength range from about 620 nm to about 655 nm and/or yellow-green light in the wavelength range from about 530 nm to about 580 nm, and directing the generated light to a target object containing an accumulation of a fluorescable biomarker, such that the generated light reaches the fluorescable biomarker and excites the fluorescable biomarker to thereby cause it to emit fluoresced light within a characteristic wavelength band. The fluoresced light from the fluorescable biomarker is captured. A digital data file representative of the captured fluoresced light is created. A representative image is formed on a display screen from the digital data file. An apparatus for generating an image on a display screen using the above method is also disclosed.

### CROSS-REFERENCE TO RELATED APPLICATIONS

This application claims priority to and the benefit of U.S. Provisional Application No. 62/699,723 filed on Jul. 17, 2018, the entirety of which is incorporated herein by reference.

### FIELD OF THE INVENTION

The present invention relates to methods of and apparatus for producing an image on a display screen, and more particularly to methods of and apparatus for producing an image on a display screen, such as a digital display screen, wherein at least one fluorescable biomarker is used in a person's body to fluoresce light.

### BACKGROUND OF THE INVENTION

One modern and effective method of analysing portions of a person's body involves administering a compound that migrates to and accumulates at the portion of the body in question. This portion of the person's body basically becomes a target object to be analysed. The compound ultimately provides a fluorescable biomarker accumulated at the target object. The compound itself may be the fluorescable biomarker, or alternatively the compound may produce the fluorescable biomarker as a derivative once metabolized in the body. One compound that is commonly used to derive a fluorescable biomarker in a person and one example of a fluorescing agent is 5-aminolevulinic acid (5-ALA) or methyl aminolevulinate (MAL), or collectively ALA, which results in production of an intracellular photosynthesizer, protoporphyrin IX (PpIX). PpIX has known light absorption and emission characteristics, and is further known to accumulate at higher levels in cancer cells than in surrounding tissue.

It is well known in the medical profession that 5-ALA, which is used in the treatment of cancer by collecting in a cancerous mass, illuminates when exposed to light. It is also known that blue light around the wavelength of 407 nm, cyan light in around the wavelength of 505 nm, green light in around the wavelength of 535 nm, yellow green light in around the wavelength of 580 nm, and red light in around the wavelength of 635 nm, all illuminate 5-ALA very readily, and better than other wavelengths of light.

Typically, the subsequent analysis involves bombarding the target object with specific wavelengths of light in order to activate the fluorescable biomarker. The fluorescable biomarker is thereby caused to fluoresce visible light, typically within a narrow band of wavelengths.

In order to assess target objects such as tumors, especially cancerous tumors, for subsequent treatment, it is necessary to capture the light that is fluoresced by the fluorescable biomarker. The captured fluoresced light can be used to form representative images of the target objects.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described herein below.

### BRIEF DESCRIPTION OF THE DRAWINGS

The novel features which are believed to be characteristic of the method of and apparatus for producing an image on a display screen according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIG. 1 is a front elevational view of the apparatus for producing an image on a display screen according to the present invention;

FIG. 2 is a cut-away side elevational view of the apparatus of FIG. 1;

FIG. 3 is a rear elevational view of the apparatus of FIG. 1;

FIG. 4 is a picture of a person having a target surface area visible on the skin of the cheek of the person;

FIG. 5 is an enlarged view of the image representative of the target object as generated using yellow-green light;

FIG. 6 is an enlarged view of the image representative of the target object as generated using red light; and,

FIG. 7 is a flow chart of the method and apparatus according to the present invention.

### DETAILED DESCRIPTION OF THE ILLUSTRATED EMBODIMENT

The present invention will be described generally and then in detail.

In accordance with one aspect of the present invention there is disclosed a novel method of producing an image on a display screen. The method comprises the steps of generating at least one wavelength of light in a selected wavelength band of red light in the wavelength range from about 620 nm to about 655 nm; directing the generated light to a target object containing an accumulation of a fluorescable biomarker, such that the generated light reaches the fluorescable biomarker and excites the fluorescable biomarker to thereby cause it to emit fluoresced light within a characteristic wavelength band; capturing the fluoresced light from the fluorescable biomarker due to the excitation of the fluorescable biomarker; creating a digital data file representative of the captured fluoresced light; and forming a representative image on a display screen from the digital data file.

In accordance with another aspect of the present invention there is disclosed a novel apparatus for generating an image on a display screen. The apparatus comprises a source of light for generating at least one wavelength of light in a selected wavelength band of red light in the wavelength range from about 620 nm to about 655 nm, for directing the generated light to a target object containing an accumulation of a fluorescable biomarker, such that the generated light reaches the fluorescable biomarker and excites the fluorescable biomarker to thereby cause it to emit fluoresced light within a characteristic wavelength band; an optical lens for receiving the fluoresced light from the fluorescable biomarker due to the excitation of the fluorescable biomarker; a digitally operable light-capturing component for creating a digital data file representative of the captured fluoresced light; and a display screen for presenting an image representative of the digital data file.

In accordance with another aspect of the present invention there is disclosed a novel method of producing an image on a display screen. The method comprising the steps of generating at least one wavelength of light in a selected wavelength band of lime in the wavelength range from about 530 nm to about 580 nm; directing the generated light to a target object containing an accumulation of a fluorescable biomarker, such that the generated light reaches the fluorescable biomarker and excites the fluorescable biomarker to thereby cause it to emit fluoresced light within a characteristic wavelength band; capturing the fluoresced light from the fluorescable biomarker due to the excitation of the fluorescable biomarker; creating a digital data file representative of the captured fluoresced light; and forming a representative image on a display screen from the digital data file.

In accordance with another aspect of the present invention there is disclosed a novel apparatus for generating an image on a display screen. The apparatus comprising a source of light for generating at least one wavelength of light in a selected wavelength band of yellow-green light in the wavelength range from about 530 nm to about 580 nm, for directing the generated light to a target object containing an accumulation of a fluorescable biomarker, such that the generated light reaches the fluorescable biomarker and excites the fluorescable biomarker to thereby cause it to emit fluoresced light within a characteristic wavelength band; an optical lens for receiving the fluoresced light from the fluorescable biomarker due to the excitation of the fluorescable biomarker; a digitally operable light-capturing component for creating a digital data file representative of the captured fluoresced light; and a display screen for presenting an image representative of the digital data file.

In accordance with another aspect of the present invention there is disclosed a novel apparatus for generating an image on a display screen. The apparatus comprises a source of light for generating at least one wavelength of light in a selected wavelength band of light, for directing the generated light to a target object containing an accumulation of a fluorescable biomarker, such that the generated light reaches the fluorescable biomarker and excites the fluorescable biomarker to thereby cause it to emit fluoresced light within a characteristic wavelength band; an optical lens for receiving the fluoresced light from the fluorescable biomarker due to the excitation of the fluorescable biomarker; wherein the source of light comprises a plurality of light emitting diodes surrounding the optical lens to provide evenly distributed light directed to the target object; a digitally operable light-capturing component for creating a digital data file representative of the captured fluoresced light; and a display screen for presenting an image representative of the digital data file.

In accordance with another aspect of the present invention there is disclosed a novel method of producing an image on a display screen. The method comprising the steps of generating at least one wavelength of light in a selected wavelength band of light; directing the generated light to a target object containing an accumulation of a fluorescable biomarker, such that the generated light reaches the fluorescable biomarker and excites the fluorescable biomarker to thereby cause it to emit fluoresced light within a characteristic wavelength band; capturing the fluoresced light from the fluorescable biomarker due to the excitation of the fluorescable biomarker; creating a first digital data file representative of the captured fluoresced light; generating a plurality of wavelengths of light in a broad wavelength band of light; directing the generated plurality of wavelengths of light to a target surface covering said target object; capturing the re-transmitted light from the target surface; creating a second digital data file representative of the captured re-transmitted light; forming a representative image on a display screen from the first digital data file and the second digital data file.

In accordance with another aspect of the present invention there is disclosed a novel apparatus for producing an image on a display screen. The apparatus comprising a source of light for generating at least one wavelength of light in a selected wavelength band of light, and for directing the generated light to a target object containing an accumulation of a fluorescable biomarker, such that the generated light reaches the fluorescable biomarker and excites the fluorescable biomarker to thereby cause it to emit fluoresced light within a characteristic wavelength band, and for generating a plurality of wavelengths of light in a broad wavelength band of light, and for directing the generated plurality of wavelengths of light to a target surface covering said target object; an optical lens for receiving the fluoresced light from the fluorescable biomarker due to the excitation of the fluorescable biomarker and for capturing the re-transmitted light from the target surface; a digitally operable light-capturing component for creating a first digital data file representative of the captured fluoresced light and for creating a second digital data file representative of the captured re-transmitted light; and a display screen for presenting an image representative of the digital data file and the second data file.

It is also contemplated that the fluorescable biomarker could comprise Aminolevulinic acid (5-ALA) and the fluoresced light could comprise red light having wavelength around about 635 nm.

It is further contemplated that the fluorescable biomarker could comprise Aminolevulinic acid (5-ALA) and the fluoresced light could comprise red light in the wavelength range from about 620 nm to about 655 nm.

It is further contemplated that the step of generating red light in the wavelength range from about 620 nm to about 655 nm comprise generating red light in the wavelength range from about 630 nm to about 640 nm.

It is further contemplated that the step of generating red light in the wavelength range from about 630 nm to about 640 nm could comprise generating red light in the wavelength range from about 633 nm to about 637 nm.

It is further contemplated that the selected wavelength band of yellow-green light could be in the wavelength range from about 560 nm to about 575 nm.

It is further contemplated that the selected wavelength band of yellow-green light could be in the wavelength range from about 565 nm to about 570 nm.

It is known by the present inventor that red light in the wavelength range from about 620 nm to about 655 nm and yellow-green light in the wavelength range around about 530 nm to about 580 nm both readily illuminate 5-ALA disposed within a target object, and also readily penetrate subcutaneously into human tissue up to perhaps about five (5) millimetres, depending on the wavelength and the intensity of the light.

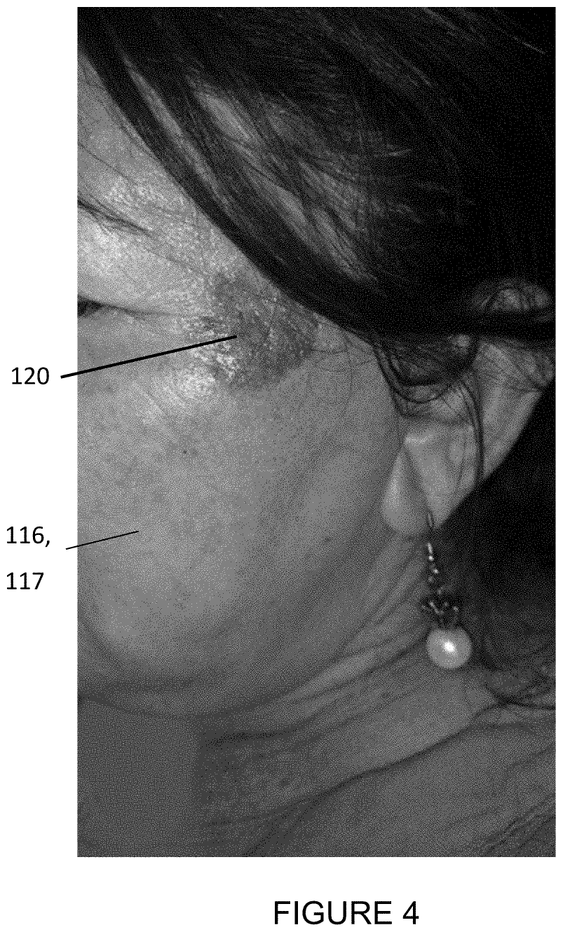

In experiments, it is been found that the fluoresced red light in the wavelength range around about 635 nm emanating from the 5-ALA in the target object, such as Leishmaniasis (as shown in FIG. 4), or perhaps a cancerous tumor, can be captured and used to form a useful image, that accurately and usefully depicts and/or indicates the size and shape and location including the subcutaneous depth of the target object 109.

Unexpectedly, it has been found by the present inventor that using red light in the wavelength range from about 620 nm to about 655 nm to excite a fluorescable biomarker and subsequently using the fluoresced light to form a representative image on a display screen, and/or using yellow-green light in the wavelength range from about 530 nm to about 580 nm to excite a fluorescable biomarker and subsequently using the fluoresced light to form a representative image on a display screen can be used to produce a high quality useful representative image 170.

It has also been found that such a representative image 170 is sufficient to perform a qualitative assessment of the extent of cutaneous and subcutaneous conditions such as infection (e.g., parasitic infection caused by Leishmania parasites) and cancer, or other biological structures in which PpIX may accumulate at higher concentrations than in neighboring tissue. This permits detection of these conditions to be carried out using simple, low-cost apparatus that is easily deployable to developing countries and requiring minimal technical training. Further, the emitted light from a target structure or area can be easily captured by a conventional, CCD (charged-couple device) or less expensive CMOS (complementary metal-oxide-semiconductor) image sensor, and superimposed over a white-light or broader-spectrum image of the same target structure or area and displayed to a clinician or the patient for diagnosis or education.

Further, one might readily expect that a portion of the red light 140r emanating from the red light emitting diodes 150r would either reflect off or be re-transmitted by the skin 116 of the person 117, and combine with the red light being fluoresced from the compound in the object 109, thereby making it very difficult, if not impossible to create a meaningful representative image from the fluoresced light. Unexpectedly, it has also been found that the red light in the wavelength range from about 620 nm to about 655 nm does not combine with the red light re-transmitted from the skin 116 of the person 117 in a manner to obscure the proper representative image.

Further, it has also been found that the use of yellow-green light in the wavelength range around about 530 nm to about 580 nm does not interfere with the fluoresced red light emanating from the 5-ALA in the target object.

Also unexpectedly, it has been found by the present inventor that using an apparatus having light emitting diodes surrounding the optical lens to provide evenly distributed light directed to a target object containing an accumulation of a fluorescable biomarker, and using an apparatus for forming a representative image on a display screen, which image represents the light from the fluorescable biomarker and also light from the target surface above the target object, also works quite well.

In all cases, illumination was provided by red light emitting diodes (LEDs) emitting at about a 635 nm wavelength at a fluence rate (at the skin surface) of about 140 J/cm² over 900 seconds exposure. It was estimated that fluence at a 2 mm depth into skin was about 20% of the surface fluence rate. 635 nm images were captured using a CMOS camera in an inexpensive smartphone (Alcatel® Pixi 4 (6), TCL Communication Technology Holdings Limited, Guangdong, China) and two 75-micron thicknesses of a dye-coated polyethylene terephthalate color effect lighting filter in #106 Primary Red from Rosco Laboratories, Inc., Stamford, Conn., USA.

While it may be expected that reflection of impinging red light on the patient's skin would combine with emitted red light, rendering it difficult to discern the fluorescence from the accumulated PpIX, it was found that a useful representative image could still be formed from the red light resulting from illumination of the patient's skin surface. The captured red light is believed to be emitted light in the 635 nm range.

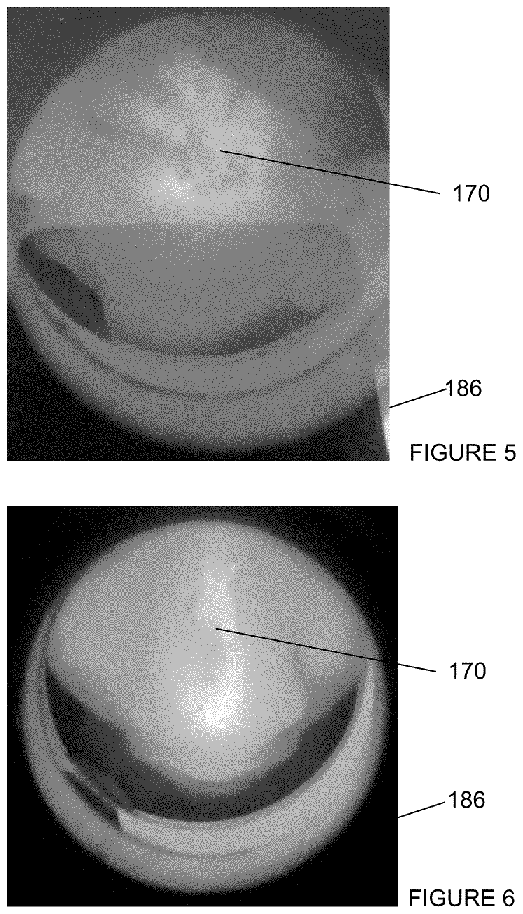

Excitation and detection may be carried out using a single portable, dual-purpose apparatus, or alternatively by providing an illumination apparatus that can be mounted to a general-purpose electronic device provided with an image sensor, such as a smartphone. The latter is depicted in FIGS. 5-7 with an illumination apparatus 100 mounted on a digital electronic device 184 (in this example, a smartphone), but it will be appreciated by those skilled in the art that with suitable modification, the illumination apparatus 100 and the necessary components of the digital electronic device 184 (e.g., image sensor, microprocessor, display screen) may be combined in a unitary structure.

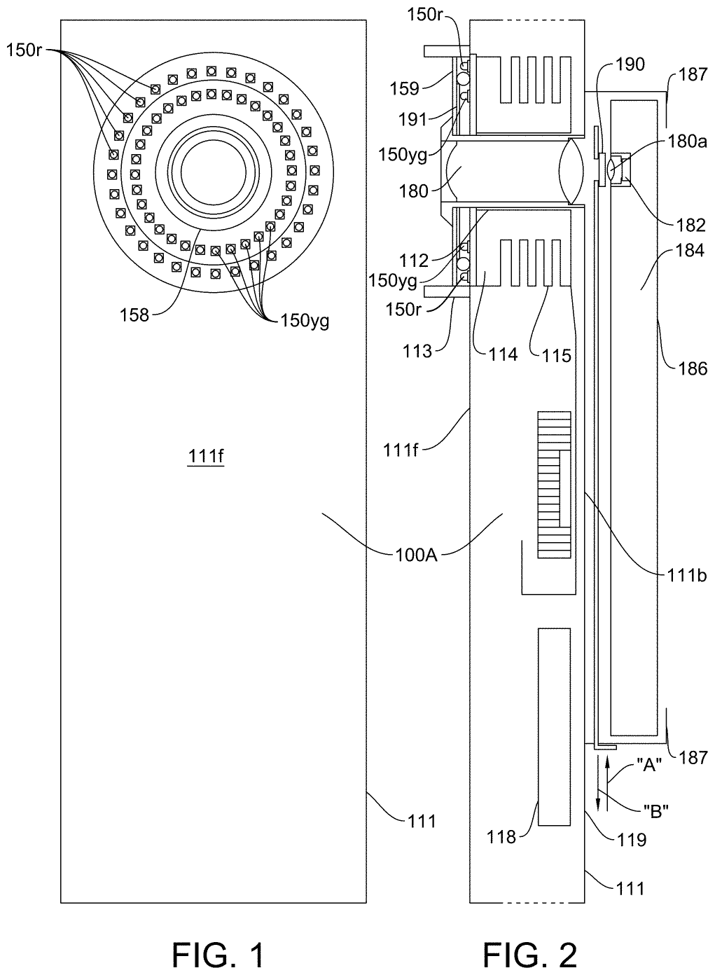

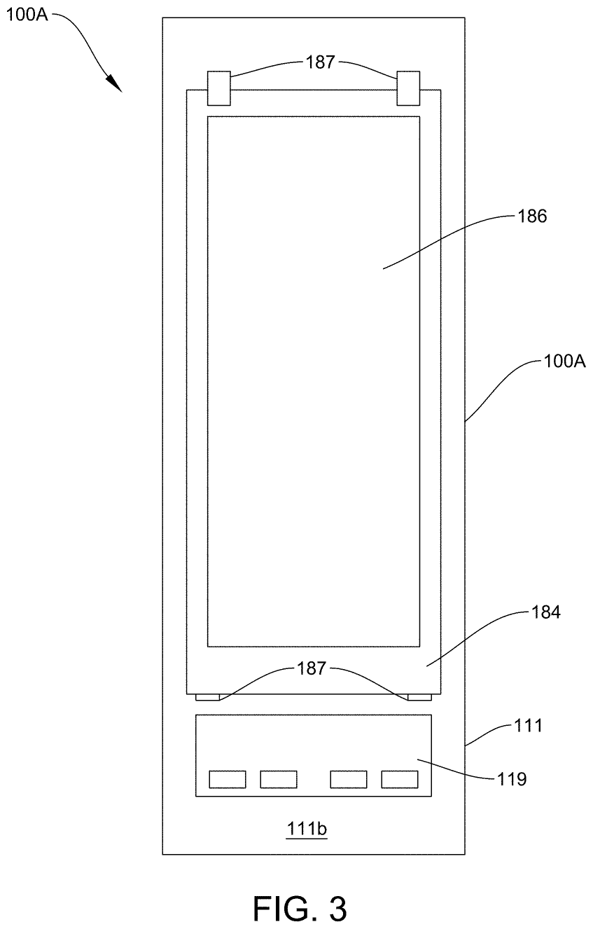

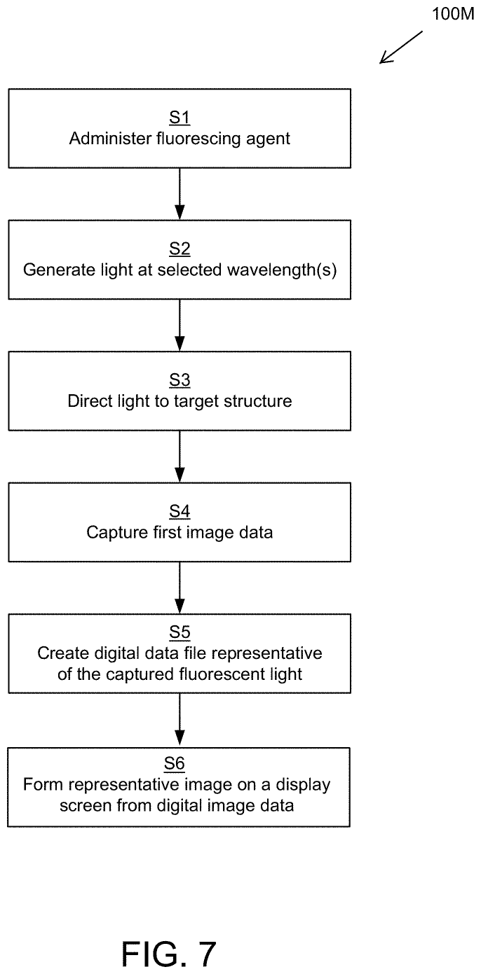

Reference will now be made to FIGS. 1 through 7, which show an embodiment of the method of generating an image of a target object according to the present invention, as indicated by general reference numeral 100M (see FIG. 7), and an apparatus for generating an image of a target object according to the present invention, as indicated by general reference numeral 100A.

In referring to FIGS. 1 through 7 of the drawings, it will be noted that these figures illustrate an embodiment of the method of generating an image of a target object according to the present invention and an apparatus for generating an image of a target object according to the present invention. A surface portion of the target object is best seen in FIG. 4.

More specifically, the first embodiment of the method of generating an image of a target object 120 according to the present invention comprises the following steps as set forth below, and as illustrated in FIG. 1. The method is indicated by the general reference numeral 100M (see FIG. 7).

An initial step, as indicated by reference numeral 102, is that of administering a fluorescable biomarker 130, such as fluorescable biomarker 130, to a person such that the fluorescable biomarker 130 at least partially accumulates at the target object 120 in order to be available to fluoresce when bombarded by light. Typically, the fluorescable biomarker 130 is administered orally, as can be seen in FIG. 5. The fluorescable biomarker 130 causes tissue fluorescence of the target object 120 after a period of time subsequent to oral administration, under the bombardment of suitable wavelengths of light, as discussed above. Typically this period of time may be about thirty (30) minutes to about twelve (12) hrs, with the period of time of perhaps three (3) to six (6) hours providing the highest concentration.

A next step, as indicated by reference numeral 104, is that of generating yellow-green light 140yg, and/or red light 140r. The yellow-green light 140yg is preferably in the wavelength range around about 530 nm to about 580 nm, or more narrowly in the wavelength range from about 560 nm to about 575 nm, or even more narrowly in the wavelength range from about 565 nm to about 570 nm. The red light 140r is preferably in the wavelength range around 620 nm to about 655 nm, or more narrowly in the wavelength range from about 630 nm to about 640 nm, or even more narrowly in the wavelength range from about 633 nm to about 637 nm.

The steps of generating yellow-green light 140yg and generating red light 140r may favorably be done exteriorly to the person's body 117. Further, the steps of generating yellow-green light 140yg and generating red light 140r may favorably be done by a plurality of light emitting diodes (LEDs) 150. More specifically, there are yellow-green light emitting diodes 150yg that generate the yellow-green light 140yg, and red light emitting diodes 150r that generate the red light 140r. The yellow-green light emitting diodes (LEDs) 150yg are arranged in an inner circumferential ring of light emitting diodes. Similarly, the red light emitting diodes 150r are arranged in an outer circumferential ring of light emitting diodes that is disposed circumferentially around the inner ring of yellow-green light emitting diodes 150yg. The inner circumferential ring of yellow-green light emitting diodes 150yg and the outer circumferential ring of red light emitting diodes 150r are separated one from the other by an annular light barrier 158.

It has been found that LEDs by Phillips Lighting of Eindhoven, Netherlands, and under license to Lumileds of Schipol, Netherlands, under the general name of limelight LEDs, some of which are marketed as LED LUXEON Z LIME 568NM 2SMD, produce very suitable yellow-green light in the wavelength range from about 530 nm to about 580 nm.

The light emitting diodes 150 may produce a light intensity of about 50 mW/cm² to about 1000 mW/cm², may produce a light pattern on a person's skin 116 over a surface area of about 30 cm², and may draw an electrical power of about 40 watts to about 100 watts.

A next step, as indicated by the reference numeral 106, is that of directing the generated yellow-green light 140yg and the generated red light 140r to the area of the person's skin 116 over the target object 120 such that the yellow-green light 140yg and the red light 140r penetrate subcutaneously to reach the target object 120. It has been found that the red light 140r penetrates even more deeply than the yellow-green light 140yg.

A next step, as indicated by the reference numeral 108, is that of capturing the light fluoresced by the target object 120 due to the excitation of the fluorescable biomarker 130 by the yellow-green light 140yg and the red light 140r to thereby form an image 170.

The step of capturing the light fluoresced by the target object 120 due to the excitation of the fluorescable biomarker 130 by the by the yellow-green light 140yg and the red light 140r to thereby form the image 170 may comprise using an optical lens 180. The step of capturing the light fluoresced by the target object 120 due to the excitation of the fluorescable biomarker 130 by the by the yellow-green light 140yg and the red light 140r to thereby form the image 170 may additionally comprise using a digitally operable light-capturing component 182, wherein the digitally operable light-capturing component 182 may comprise a charge-coupled device (CCD) or a CMOS image sensor. The charge-coupled device (CCD) or CMOS image sensor 182 may be part of a digital electronic device 184 having a display screen 186. Further, the digital electronic device 184 may comprise a cellular telephone 184 (smart phone) that includes camera hardware and software, as illustrated, or may comprise a suitable digital camera (not specifically shown). It has been found that the use of smart phone is highly desirable since many apps are available to permit control of the camera function of suitable smart phones.

Further, as readily seen in the Figures, the light emitting diodes 150 may be disposed circumferentially around the optical lens 180. As can be seen in FIG. 1, the red light emitting diodes 150r and disposed circumferentially around the yellow-green light emitting diodes 150yg. It has been shown that using concentric rings of LEDs is an effective way to produce evenly disbursed light.

A clear uncolored window 159 is disposed in protective relation in front of the LEDs 150. Additionally, a yellow-green light filter 191 may be disposed in front of the yellow-green light emitting diodes 150yg in order to filter out any small amounts of red light that might emanate from lime LEDS. The annular light barrier 158 precludes light from the yellow-green light emitting diodes 150yg from directly shining outwardly past the yellow-green light filter 191 and therefore must pass through the yellow-green light filter 191.

A further step may be that of filtering the light fluoresced by the target object 120, and may comprise using a red light optical filter 190. Favourably, the optical filter 190 passes light in the wavelength range of about 600 nm-900 nm, and even more favourably passes light in the wavelength range of about 600 nm-650 nm.

In another aspect, the present invention comprises an apparatus 110 for generating an image of a target object, as can perhaps be best seen in FIGS. 1 through 3. The apparatus comprises a housing 111, having a front 111f, a back 111b, a throughpassage 112 for the optical lens 180, and an annular light shade 113 around the front of the optical lens 180. The apparatus further comprises a source of yellow-green light 140yg in the wavelength range wavelength around about wavelength range from about 530 nm to about 580 nm and a source of red light in the wavelength range from about 620 nm to about 655 nm. The source 150 of yellow-green light 140yg comprises yellow-green light emitting diodes 150yg. The source 150 of red light 140r comprises red light emitting diodes 150r.

A fan 114 and a heat sink 115 are disposed sin surrounding relation around the optical lens 180 for cooling the light emitting diodes 150. The apparatus also comprises an electronic circuit 118 for driving the various electrical and electronic components. A electronic control interface 119 is disposed on the housing 111 below the bottom end of the smart phone 184 for permitting for convenient control of the apparatus 100A.

The optical lens 180, specifically a very wide angle (a.k.a. fisheye) type optical lens 180, is for capturing the light fluoresced by the target object due to the excitation of 5-ALA by the green light to thereby form an image 170. The digitally operable light-capturing component 182 captures the light fluoresced by the target object 120 due to the excitation of the fluorescable biomarker 130 by the yellow-green light 140yg and the red light 140r, to thereby form the image 170. The optical lens 180 focuses the received light onto the digitally operable light-capturing component 182, to thereby form from the captured light the image 170 representative of the target object.

The digitally operable light-capturing component 183 may comprise a charge-coupled device (CCD) or a CMOS image sensor. The charge-coupled device (CCD) or CMOS image sensor 182 may be part of a digital electronic device 184 having a display screen 186. Further, the digital electronic device 184 may comprise a cellular telephone 184 (smart phone) that includes camera hardware and software, as illustrated, or may comprise a suitable digital camera (not specifically shown). It has been found that the use of smart phone is highly desirable since many apps are available to permit control of the camera function of suitable smart phones. The digital electronic device 184 may alternatively be a conventional digital camera (not specifically show). It should also be understood that the digital electronic device 184 may be used to produce and save either or both of still images and or moving images. A clip-type frame 187, is used to retain the digital electronic device 184 (smart phone or camera) in place. Customized adapters (not specifically shown) could also be used in conjunction with the clip-type frame 187. It should be noted that the digital electronic device 184 may also have its own built-in a supplemental lens 180a.

The optional red light filter 190 is mounted in sliding engagement between the back end of the optical lens 180 and the camera lens 180a of the smart phone 184. The red light filter 190 may be slid into place, as indicated by arrow “A”, and out of place, as indicated by arrow “B”. Accordingly, the red light filter 190 may be selectively employed as required, in the event that the red light passing through the optical lens 180 contains contaminants, or in other words undesirable wavelengths of light.

The light emitting diodes 150 may produce a light intensity of about 50 mW/cm2 to about 1000 mW/cm2, may produce a light pattern on a person's skin 116 over a surface area of about 30 cm2, and may draw an electrical power of about 40 watts to about 100 watts.

The present invention also comprises, by using one or more of these wavelengths of light as discussed above, the step of generating multiple images of target objects from the same external location on person's skin 116. The yellow-green light 140yg in the wavelength range from about 530 nm to about 580 nm and the red light 140r in the wavelength range from about 620 nm to about 655 nm are generated separately and used separately to each form an image at an accurately estimated subcutaneous depth. More specifically, the image generated by the yellow-green light 140yg is shown in FIG. 5, and the image generated by the red light 140r is shown in FIG. 6.

These images are representative of the fluorescence of light at different depths within a person's body beneath the person's skin 116. The approximate depth of penetration of each of the wavelengths of light into human tissue is pre-determined by previous measurement. These known penetration measurements are used to predict the depth of the target object 120, or portion thereof, that is being represented by the captured images.

The steps include generating at least one wavelength of light in a selected wavelength band of light and generating a plurality of wavelengths of light in a broad wavelength band of light, as can be seen in step S2 in FIG. 7. The at least one wavelength of light in a selected wavelength band of light may be any suitable wavelength. It has been found that red light in the wavelength range from about 620 nm to about 655 nm and yellow-green light in the wavelength range from about 530 nm to about 580 nm can be used to produce a clear representative image 170.

Further steps include directing the generated light of at least one wavelength to the target object 120 containing an accumulation of a fluorescable biomarker 130, as can be seen in step S2 in FIG. 7, such that the generated light reaches the fluorescable biomarker 130 and excites the fluorescable biomarker 130 to thereby cause it to emit fluoresced light within a characteristic wavelength band. If 5-ALA is used to derive the fluorescable biomarker 130, the fluoresced red light is in the wavelength around about 635 nm emanating from the fluorescable biomarker 130 in the target object 120.

The next steps include capturing the fluoresced light from the fluorescable biomarker 130 accumulated in the target object 120 and capturing the re-transmitted light and/or reflected light from the target surface 124, as can be seen in step S4 in FIG. 7. In the embodiment of the present invention discussed above, the wavelength of the fluoresced light is around about 635 nm due to the excitation of the fluorescable biomarker 130.

Subsequently, the next step is to create a first digital data file 161 representative of the captured fluoresced light, as can be seen in step S5 in FIG. 7. The first digital data file 161 can be stored in any suitable computer memory medium 185 within the digital electronic device 184.

Once the first digital data file 161 has been created, the next step comprises forming a representative image 171 on a display screen from the first digital data file 161 and the second digital data file 162, as can be seen in step S6 in FIG. 7.

In another aspect, the present invention comprises an apparatus comprising a source of light 140 for generating at least one wavelength of light in a selected wavelength band of light. As illustrated, the source 150 of light 140 comprises a source of yellow-green light 140yg in the wavelength range wavelength around about wavelength range from about 530 nm to about 580 nm, namely yellow-green light emitting diodes 150yg, and a source of red light 150r in the wavelength range from about 620 nm to about 655 nm, namely red light emitting diodes 150r.

The digital electronic device 184 is used, typically via manually manipulation, to direct the generated light of at least one wavelength to the target object 120 containing an accumulation of a fluorescable biomarker 130, such that the generated light reaches the fluorescable biomarker and excites the fluorescable biomarker to thereby cause it to emit fluoresced light within a characteristic wavelength band. The digital electronic device 184 may also be used, typically via manually manipulation, to direct the plurality of wavelengths of light in a broad wavelength band of light to the target surface 124 over the target object 120.

The optical lens 180 is for receiving the fluoresced light from the fluorescable biomarker 130 due to the excitation of the fluorescable biomarker and for capturing the re-transmitted light from the target surface, as described above.

The digitally operable light-capturing component 182, which may comprise a charge-coupled device (CCD) or a CMOS image sensor, as described above, is for creating the first digital data file 161 representative of the captured fluoresced light and for creating a second digital data file 162 representative of the captured re-transmitted light from the target surface 124.

The display screen 186, as discussed above, which is part of the digital electronic device 184, is for presenting the representative image 171 that is representative of the digital data file 161 and the second data file 162, as discussed above.

Other variations of the above principles will be apparent to those who are knowledgeable in the field of the invention, and such variations are considered to be within the scope of the present invention. Further, other modifications and alterations may be used in the design and manufacture of the impact mechanism of the present invention without departing from the spirit and scope of the accompanying claims.

1. A method of producing an image on a display screen, said method comprising the steps of:
generating at least one wavelength of light in a selected wavelength band of red light in the wavelength range from about 620 nm to about 655 nm;
directing the generated light to a target object containing an accumulation of a fluorescable biomarker, such that the generated light reaches the fluorescable biomarker and excites the fluorescable biomarker to thereby cause it to emit fluoresced light within a characteristic wavelength band;
capturing the fluoresced light from the fluorescable biomarker due to the excitation of the fluorescable biomarker;
creating a digital data file representative of the captured fluoresced light; and,
forming a representative image on a display screen from said digital data file.

2. The method of producing an image on a display screen according to claim 1, wherein said fluorescable biomarker comprises Aminolevulinic acid (5-ALA) and the fluoresced light comprises red light having wavelength around about 635 nm.

3. The method of producing an image on a display screen according to claim 1, wherein said fluorescable biomarker comprises Aminolevulinic acid (5-ALA) and the fluoresced light comprises red light in the wavelength range from about 620 nm to about 655 nm.

4. The method of producing an image on a display screen according to claim 1, wherein the step of generating red light in the wavelength range from about 620 nm to about 655 nm comprises generating red light in the wavelength range from about 630 nm to about 640 nm.

5. The method of producing an image on a display screen according to claim 4, wherein the step of generating red light in the wavelength range from about 630 nm to about 640 nm comprises generating red light in the wavelength range from about 633 nm to about 637 nm.

6. An apparatus for generating an image on a display screen, said apparatus comprising:
a source of light for generating at least one wavelength of light in a selected wavelength band of red light in the wavelength range from about 620 nm to about 655 nm, and for directing the generated light to a target object containing an accumulation of a fluorescable biomarker, such that the generated light reaches the fluorescable biomarker and excites the fluorescable biomarker to thereby cause it to emit fluoresced light within a characteristic wavelength band;
an optical lens for receiving the fluoresced light from the fluorescable biomarker due to the excitation of the fluorescable biomarker;
a digitally operable light-capturing component for creating a digital data file representative of the captured fluoresced light; and,
a display screen for presenting an image representative of said digital data file.

7. The apparatus for generating an image on a display screen according to claim 6, wherein said fluorescable biomarker comprises Aminolevulinic acid (5-ALA) and the fluoresced light comprises red light having wavelength around about 635 nm.

8. The apparatus for generating an image on a display screen according to claim 6, wherein said fluorescable biomarker comprises Aminolevulinic acid (5-ALA) and the fluoresced light comprises red light in the wavelength range from about 620 nm to about 655 nm.

9. The apparatus for generating an image on a display screen according to claim 6, wherein the selected wavelength band of red light is in the wavelength range from about 630 nm to about 640 nm.

10. The apparatus for generating an image on a display screen according to claim 9, wherein the selected wavelength band of red light is in the wavelength range from about 633 nm to about 637 nm.

11. A method of producing an image on a display screen, said method comprising the steps of:
generating at least one wavelength of light in a selected wavelength band of yellow-green light in the wavelength range from about 530 nm to about 580 nm;
directing the generated light to a target object containing an accumulation of a fluorescable biomarker, such that the generated light reaches the fluorescable biomarker and excites the fluorescable biomarker to thereby cause it to emit fluoresced light within a characteristic wavelength band;
capturing the fluoresced light from the fluorescable biomarker due to the excitation of the fluorescable biomarker;
creating a digital data file representative of the captured fluoresced light; and, forming a representative image on a display screen from said digital data file.

12. The method of producing an image on a display screen according to claim 11, wherein said fluorescable biomarker comprises Aminolevulinic acid (5-ALA) and the fluoresced light comprises red light having wavelength around about 635 nm.

13. The method of producing an image on a display screen according to claim 11, wherein said fluorescable biomarker comprises Aminolevulinic acid (5-ALA) and the fluoresced light comprises red light in the wavelength range from about 620 nm to about 655 nm.

14. The method of producing an image on a display screen according to claim 11, wherein the step of generating yellow-green light in the wavelength range from about 530 nm to about 580 nm comprises generating yellow-green light in the wavelength range from about 560 nm to about 575 nm.

15. The method of producing an image on a display screen according to claim 14, wherein the step of generating yellow-green light in the wavelength range from about 560 nm to about 575 nm comprises generating yellow-green light in the wavelength range from about 565 nm to about 570 nm.

16. An apparatus for generating an image on a display screen, said apparatus comprising:
a source of light for generating at least one wavelength of light in a selected wavelength band of yellow-green light in the wavelength range from about 530 nm to about 580 nm, and for directing the generated light to a target object containing an accumulation of a fluorescable biomarker, such that the generated light reaches the fluorescable biomarker and excites the fluorescable biomarker to thereby cause it to emit fluoresced light within a characteristic wavelength band;
an optical lens for receiving the fluoresced light from the fluorescable biomarker due to the excitation of the fluorescable biomarker;
a digitally operable light-capturing component for creating a digital data file representative of the captured fluoresced light; and, a display screen for presenting an image representative of said digital data file.

17. The apparatus for generating an image on a display screen according to claim 16, wherein said fluorescable biomarker comprises Aminolevulinic acid (5-ALA) and the fluoresced light comprises red light having wavelength around about 635 nm.

18. The apparatus for generating an image on a display screen according to claim 16, wherein said fluorescable biomarker comprises Aminolevulinic acid (5-ALA) and the fluoresced light comprises red light in the wavelength range from about 620 nm to about 655 nm.

19. The apparatus for generating an image on a display screen according to claim 16, wherein the selected wavelength band of yellow-green light is in the wavelength range from about 560 nm to about 575 nm.

20. The apparatus for generating an image on a display screen according to claim 19, wherein the selected wavelength band of yellow-green light is in the wavelength range from about 565 nm to about 570 nm.

21. An apparatus for generating an image on a display screen, said apparatus comprising:
a source of light for generating at least one wavelength of light in a selected wavelength band of light, and for directing the generated light to a target object containing an accumulation of a fluorescable biomarker, such that the generated light reaches the fluorescable biomarker and excites the fluorescable biomarker to thereby cause it to emit fluoresced light within a characteristic wavelength band;
an optical lens for receiving the fluoresced light from the fluorescable biomarker due to the excitation of the fluorescable biomarker;
wherein said source of light comprises a plurality of light emitting diodes surrounding said optical lens to provide evenly distributed light directed to said target object;
a digitally operable light-capturing component for creating a digital data file representative of the captured fluoresced light; and,
a display screen for presenting an image representative of said digital data file.

22. The apparatus for generating an image on a display screen according to claim 21, wherein said light emitting diodes emit a wavelength band of yellow-green light in the wavelength range from about 530 nm to about 580 nm.

23. The apparatus for generating an image on a display screen according to claim 21, wherein said light emitting diodes emit a wavelength band of red light in the wavelength range from about 620 nm to about 655 nm.

24. The apparatus for generating an image on a display screen according to claim 21, wherein said light emitting diodes comprise both light emitting diodes that emit a wavelength band of yellow-green light in the wavelength range from about 530 nm to about 580 nm and light emitting diodes that emit a wavelength band of red light in the wavelength range from about 620 nm to about 655 nm.

25. The apparatus for generating an image on a display screen according to claim 21, wherein said light emitting diodes that emit a wavelength band of yellow-green light in the wavelength range from about 530 nm to about 580 nm and light emitting diodes that emit a wavelength band of red light in the wavelength range from about 620 nm to about 655 nm are situated in an alternating pattern around said optical lens.
